SEQUENCE LISTING (3 sequences)

SEQ ID NO: 1 - protein, 29 aa, Artificial Sequence
  Synthetic Polypeptide
KGRQYIKANSKFIGITERGRILKEPVHGV

SEQ ID NO: 2 - protein, 27 aa, Artificial Sequence
  Synthetic Polypeptide
LEESQVNDDIFNSLVKSVQQEQQHNVK

SEQ ID NO: 3 - protein, 25 aa, Artificial Sequence
  Synthetic Polypeptide
ERRAKEKLQEQQSDLEQRKADTKKK

(19) United States
(12) Patent Application Publication
(10) Pub. No.: US 2003/0049267 A1
(43) Pub. Date: Mar. 13, 2003

(54) Systemic immune response induced by mucosal administration of lipid-tailed polypeptides without adjuvant

(75) Inventors: Pierre Druilhe, Paris (FR); Helene Gras-Masse, Merignies (FR); Lbachir Benmohamed, Montereau (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris Cedex (FR)

(21) Appl. No.: 10/206,535

(22) Filed: Jul. 29, 2002

Related U.S. Application Data
(62) Division of application No. 09/732,754, filed on Dec. 11, 2000.
(60) Provisional application No. 60/169,952, filed on Dec. 9, 1999.

(51) Int. Cl. A61K 39/00

(52) U.S. Cl. 424/185.1

(57) ABSTRACT

A method of inducing an immune response by applying an immune response inducing effective amount of a lipopeptide to a mucosal membrane of a subject.

This application claims priority under 35 U.S.C. §119(e) to U.S. provisional application serial No. 60/169,952, filed Dec. 9, 1999.

### BACKGROUND OF THE INVENTION

1. Field of the Invention

The present invention relates to methods of stimulating an immune response by applying lipid-modified polypeptides to mucosal membranes.

2. Description of the Background

Immunization via an application to mucosal surfaces, without adjuvant and without a physical penetration by needles, would greatly increase the ease of vaccination. Recent advances in vaccinology have created an array of novel living and nonliving Ag-delivery systems, and intriguing adjuvants that can be administrated via mucosal routes (reviewed by Levine and Dougan, 1998; Michalek et coll., 1999; Hantman et coll., 1999). The trivalent attenuated Sabin poliovirus vaccine and the cornerstone of the global poliomyelitis-eradication program, has encouraged the development of other orally or nasally delivered living vaccines (Sabin, 1985). A promising way of delivering Ags to the mucosal surface and stimulating systemic immune responses is the use of attenuated bacteria (Salmonella typhi, Shigella, V cholerae, Yersinia, Escherichia coli, BCG, Lactobacillus and Streptococcus gordoni) or viruses (i.g. adenoviruses or poliovirus), that are capable of infecting or colonizing mucosal surface, and to express the desired heterologous Ags (reviewed by Nataro and Levine, 1999). While mucosal immunization with live-attenuated bacteria and viruses was shown to be effective at inducing systemic immune responses, this strategy may be limited by safety issues. Therefore, there is interest in the development of a non-living, safe mucosal vaccine.

Mucosal immunization by sub-unit recombinant vaccines or polypeptides, initially aimed at inducing local immunity, has proven a major challenge (Nardelli et coll., 1994; Mannino et coll., 1995). This has been hampered in practical terms by the obstacles of poor adsorption to mucosal membranes and poor immunogenicity, coupled with a paucity of sufficiently potent adjuvants that can be tolerated by humans. The cholera toxin (CT) produced by the bacterium Vibrio cholera and the closely related heat-labile enterotoxin (LT) of E. coli and their B subunits (CTB and LTB) are commonly used experimentally as mucosal adjuvants that indeed augrnent the local and systemic immune responses to polypeptides or protein Ags (Snider, 1995 ; Porgador et coll., 1997). Because of their severe diarrheagenic property when ingested by human beings, in amounts as low as 0.5 mg, these toxins, as well as genetically modified attenuated derivatives, are unfortunately unacceptable for human use (Di Tommaso et coll., 1996).

Recently, we and others have established that parenteral injections of soluble lipopeptides can induce, without adjuvant, systemic B, T helper and cytotoxic T cell (CTL) responses (Bourgault et coll. 1994 and 1997 ; BenMohamed et coll. 1997 ; Perlaza et coll., 1998 ; Vitiello et coll., 1995 ; Livingston et coll., 1997 ; Mortara et coll., 1998; BenMohamed et coll., submitted). The mechanisms by which lipid-tailed polypeptides induce in vivo B and T cell responses are not yet fully understood and have been the subject of several speculations (BenMohamed et coll., 1997). The palmitic part of lipopeptides may be able to attach and fuse to the lipidic component of cell membranes and to deliver the lipopeptide into the cytoplasm: Palmitoyl-polypeptides of 8-40 residues are able to passively cross the cell membrane of non-phagocytic cells to modulate the activity of intracytoplasmic targets, such as protein Kinase C (Thiam et coll., 1997), or integrins (Stephens et coll., 1998) or cytoplasmic domains of EFN-gamma receptors (Thiam et coll., 1998, J999). This process extends to biological barriers thicker than cell membranes as monoacylation of a 14 Kd enzyme enables its transport across an in vitro model of the blood brain barrier (Chopineau et coll, 1998).

Synthetic lipid-tailed polypeptides, derived from the P. falciparum LSA-1 and LSA-3 proteins (Fidock et coll., 1994; Daubersies et coll. submitted), which B and T cell immunogenicity by S.C. route has been well established in both mice and non-human primates (BenMohamed et coll. 1997; Perlaza et coll., 1998; BenMohamed et coll., submitted), were administered sub-lingually (S.L.) and intranasally (I.N.) in mice. Polypeptide and parasite systemic specific B and T cell responses were determined to probe the transmucosal delivery of the immunogen, their characteristic and their magnitude were compared to those induced by the potent sub-cutaneous protocol. Our data revealed that i) Simple instillation of malaria lipid-tailed polypeptides to the nasal and buccal cavities without a mucosal adjuvant, results in their efficient delivery to the immune system, as evidenced by polypeptide- and parasite-specific serum antibody production (IgG) and T helper lymphocytes (Th) responses in distant lymph nodes and spleen. ii) Systemic immune responses induced by this means were found to be at least as intense, and sometimes greater than responses induced by subcutaneous route. iii) An important finding is that the route influenced the type of immune response :mucosal immunization with lipid-tailed polypeptides promoted preferentially a Th1-like immune response, whereas subcutaneous injection induced a Th2-like immune response. The use of lipid-modified polypeptides to modulate the immune system has also been described by Boutillon et al, see U.S. Pat. No. 5,871,746 and EP 0 491 628 B1.

### SUMMARY OF THE INVENTION

The capacity of lipidated-polypeptide to passively cross the plasma membrane of various cells or the blood-brain barrier as now been documented by several independent groups. We thus reasoned that the lipidation of polypeptides might also confer them the ability to cross at least the first layers of mucosal membranes, and to deliver an antigen to the immune system.

Thus, the present invention provides a method of inducing an immune response, by the delivering of an effective amount of a lipid-tailed polypeptide, also referred to as lipopolypeptide herein or lipoprotein, to a mucosal membrane of a subject.

Using antigen-specific T-helper cell responses and the production of serum antibodies to probe the immune response, we now show that intranasal or sub-lingual immunization with lipid-tailed polypeptides could represent an interesting alternative to the parenteral route strong systemic immune responses were observed, which were compared to the immune responses obtained in parallel experiments in which the same immunogen was delivered by subcutaneous route. Qualitative differences were observed when comparing parenteral or transmucosal immunization routes, with a dominant IgG1 observed after parenteral immunization versus a preferential IgG2a isotype response for the mucosal route, suggesting that distinct antigen presenting cells were involved. Mucosal immunization by lipidated polypeptides appears therefore as a novel, cost-effective and noninvasive approach that does not require the use of extraneous adjuvant.

### BRIEF DESCRIPTION OF THE FIGURES

A more complete appreciation of the invention and many of the attendant advantages thereof will be readily obtained as the same becomes better understood by reference to the following detailed description when considered in connection with the accompanying figures, wherein:

FIG. 1: Polypeptide specific antibody response induced in the serum following mucosal administration of LSA3-NRII lipid-tailed polypeptide without adjuvant. (A) Sera from individual BALB/c, C3H/HeJ, or C57BL/6 mice, immunized with the LSA3-NRII lipid-tailed polypeptide using either intranasal (black bars), sub-lingual (hatched bars) or subcutaneous route (open bars) were analyzed for polypeptide-specific total IgG antibodies using a solid-phase ELISA and compared to pre-immune sera and to sera of strain and age matched naive mice. Results are expressed as the geometric mean of individual sera in ELISA-RATIO±SD. (B) Mucosal and subcutaneous routs differ by the profile of isotypes induced. Three groups of CeH/HeJ mice were administrated either a) intranasally, b) sub-lingually, or c) subcutaneously with LSA3-NRII lipid-tailed polypeptide in saline and two weeks post-initial immunization sera were assayed for polypeptide-specific IgG1, IgG2a, IgG2b, IgG3, IgM and IgA responses. Results are expressed as individual optical density (OD₄₅₀) of sera from five mice in each group and the results are representative of three separate experiments.

FIG. 2: Mucosal immunization elicits parasite-specific antibody responses. Sera were obtained from C3H/HeJ mice at 2 weeks post-immunization by either nasal or sub-lingual route and assayed fro recognition of (a) P. falciparum sporozoites and (B) hepatic schizonts in an IFI assay as described in Material and Methods. The data are representative of three independent experiments.

FIG. 3: Systemic cellular immune responses are electited by presentation of lipid-tailed polypeptides to the nasal and sub-lingual mucosal surfaces. Groups of five C3H/HeJ mice were administrated with LSA3-NRII lipid-tailed (black bars) or non-lipidated polypeptide (hatched bars) either a) intranasally, b) sub-lingually, or c) subcutaneously. Two weeks after two administrations, cell suspensions from individual spleens were assayed for in vitro proliferation to the recall polypeptide. Results are expressed as Δ cpm. The background cpm, in unstimulated cells were 1548 fro intranasal, 2356 for sub-lingual and 1965 for subcutaneous routs. Bars represent the mean Δ cpm ± SD in each group. The data were similar and are representative of three separate experiments.

FIG. 4: T Lymphocyte responses in vitro after immunization via mucosal administration by lipopolypeptide TT-pol. (HIV). The bar represents the threshold of significance. The shift towards the left of the bar represents the quantity of response obtained. The maximum response is given by the positive control CON A (Concanavaline A).

### DETAILED DESCRIPTION OF THE INVENTION

The present invention provides a method of inducing an immune response by the delivering on an effective amount of a lipopolypeptide or a lipidated protein to a mucosal membrane of a subject. The term “lipid-tailed polypeptide” or “lipopolypeptide” refers to a polypeptide which is linked to a lipid group. The lipopeptide may have at least one lipid coupled to the (α-NH₂ and/or an ε-NH₂ functional group of the polypeptide. The lipid may be a fatty acid having from, for example, 8 to 30 carbon atoms. In a preferred embodiment, the lipid is a palmitoyl residue having 16 carbon atoms and designed hereinafter as “PAM” in the present invention.

A polypeptide as used herein is a protein or peptide having at least 10 amino acids wherein the amino acids may be modified or not as commonly known in the art.

In another embodiment of the invention, the lipopolypeptide is applied to a intranasal or sub-lingual membrane. In another embodiment, the lipopolypeptide may be applied to the mucosal membrane without adjuvant. In yet another embodiment, the lipopolypeptide may be applied to the mucosal membrane without using a needle.

Application of the lipopolypeptide may induce a B cell response. Application of the lipopolypeptide may also induce a T cell response. Alternatively, application of the lipopolypeptide may induce a B cell and a T cell response. The B cell and/or T cell response may be systemic. The invention also encompasses a composition containing in at least a lipoprotein inducing a mucosal immune response in vivo in absence of toxic adjuvant. The adjuvant is non-toxic for the mucosal membranes.

The invention also encompasses a vaccine composition for mucosal administration containing at least one polypeptide inducing an B and/or T cell response in vivo in absence of adjuvant.

The invention also encompasses an immunogenic composition containing a lipopeptide according to invention.

The invention also encompasses a method of stimulating T-Lymphocyte responses in vitro after immunization via mucosal administration comprising the following steps:

1) Immunization of BALB/C mice by mucosal administration with a peptide tetanic toxin-pol HIV palmitic antigen.

2) Collection of ganglia sub-mandibulaires at day 15, and

3) Visualization of CTL response by labeling target cells with CFSE.

The invention also encompasses a composition containing a lipid-tailed polypeptide or peptide, said lipid-tailed peptide having at least a lipid residue bound to an epitope T amino acid sequence and optionally at least one epitope B amino acid sequence.

The invention also encompasses a method of inducing an immune response by the delivering of an effective amount of lipid-tailed protein to a mucosal membrane of a subject, wherein the lipopeptide is a lipid-tailed epitope T or a lipid-tailed epitope T covalently linked to an epitope B peptide.

The invention also encompasses a composition comprising lipid-tailed polypeptide or peptide, said lipid-tailed peptide having at least a lipid residue bound to an epitope T amino acid sequence and optionally at least one epitope B amino acid sequence.

Intranasal and Sub-lingual Delivery of LSA3-NRII Lipopolypeptide Induce Circulating Specific Antibodies

To investigate the ability of lipid-tailed polypeptides to induce systemic antibody responses using mucosal route, the LSA3-NRII lipid-tailed polypeptide was administrated either intranasally (I.N.) or sub-lingually (S.L.) in BALB/c, C3H/HeJ which were found previously to respond strongly to the epitopes of LSA3-NRII, or C57BL/6 mice which is poorly responsive. Negative and positive controls received respectively, the non-lipidated polypeptide administrated by IN and S.L. route, or the same lipid-tailed polypeptide injected subcutaneously (S.C.) without adjuvant.

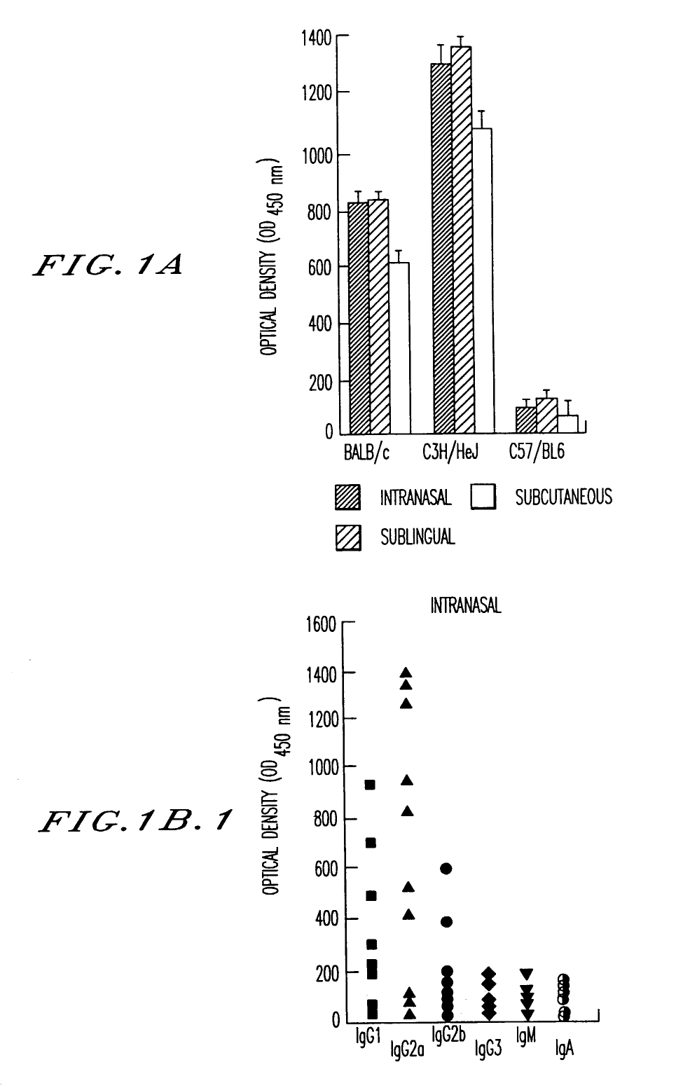

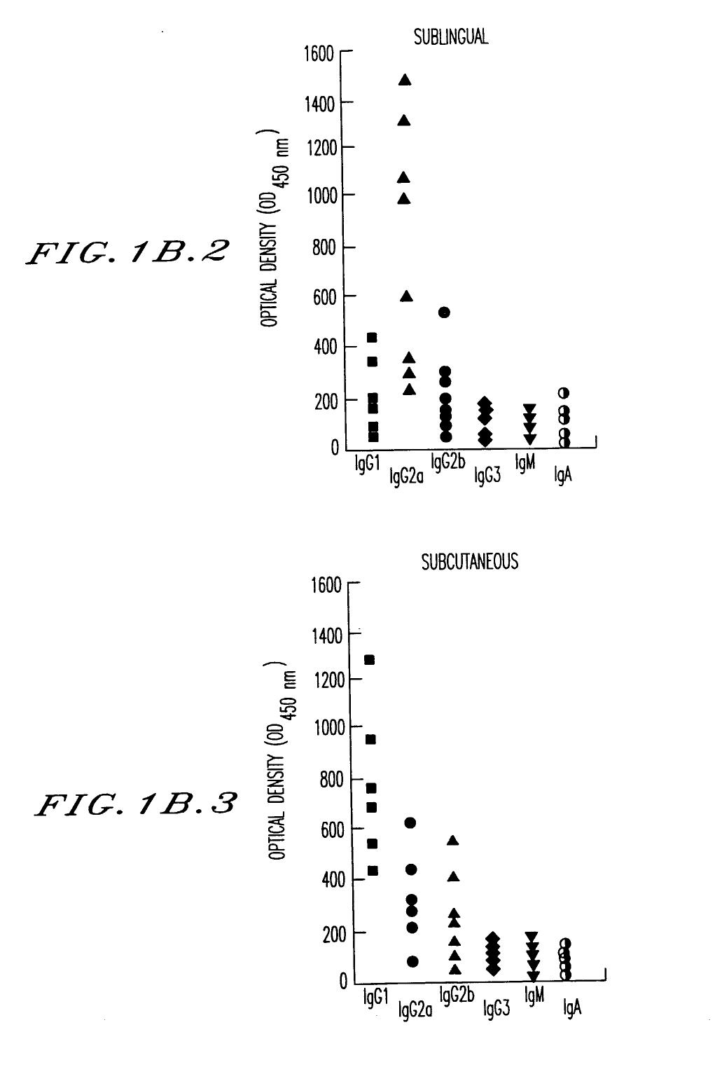

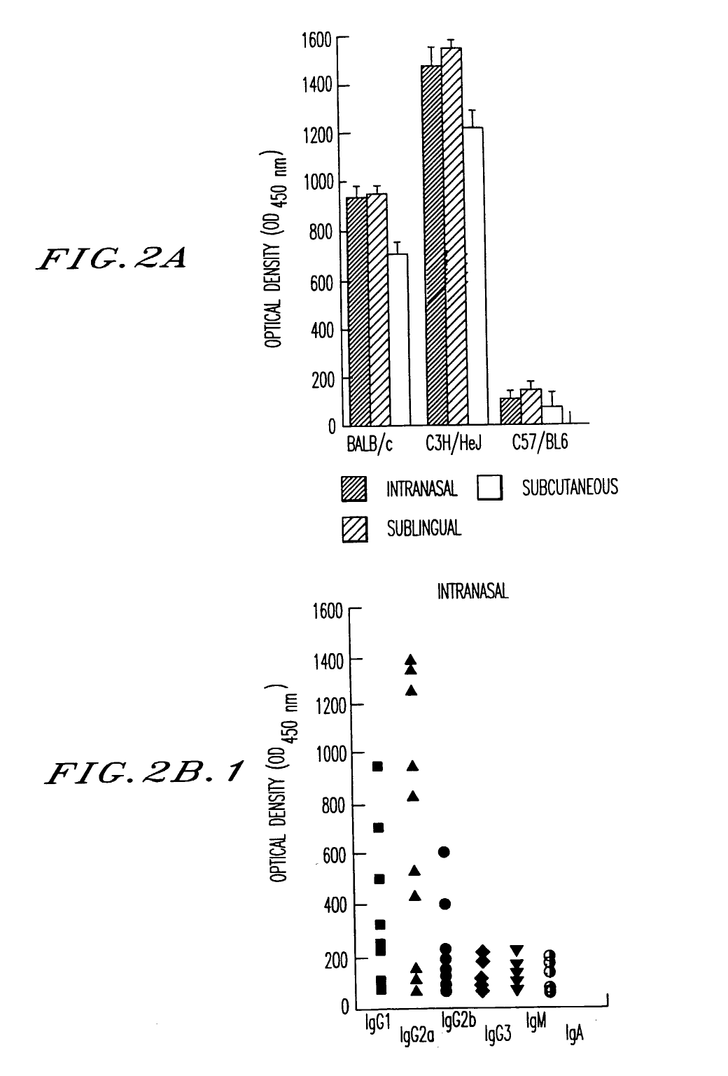

Both intranasal (I.N.) or sub-lingual (S.L.) administration were found to induce high titers of polypeptide-specific antibody responses in BALB/c and C3H/HeJ strains, measured by ELISA in serum samples taken two weeks after the second administration (FIG. 1A). The mean titers of IgG antibodies induced by both mucosal routes were found to be significantly higher than those recorded by S.C. route (p<0.05). Polypeptide specific antibody titers were significantly higher in sub-lingual immunized groups than in intranasal groups (p <0.05 in BALB/c and p<0.01 in C3H/HeJ). The antibody titers could be further enhanced by further mucosal administration of the lipid-tailed polypeptide. In contrast, using the control non-lipidated polypeptide, results were not significantly different from the preimmunization levels, ie. negative (ELISA-RATIO=0.9 in C3H/HeJ, 0.8 in BALB/c). No antibody response was found in C57BL/6 strain after either mucosal or subcutaneous administration of the LSA3-NRII lipid-tailed polypeptide.

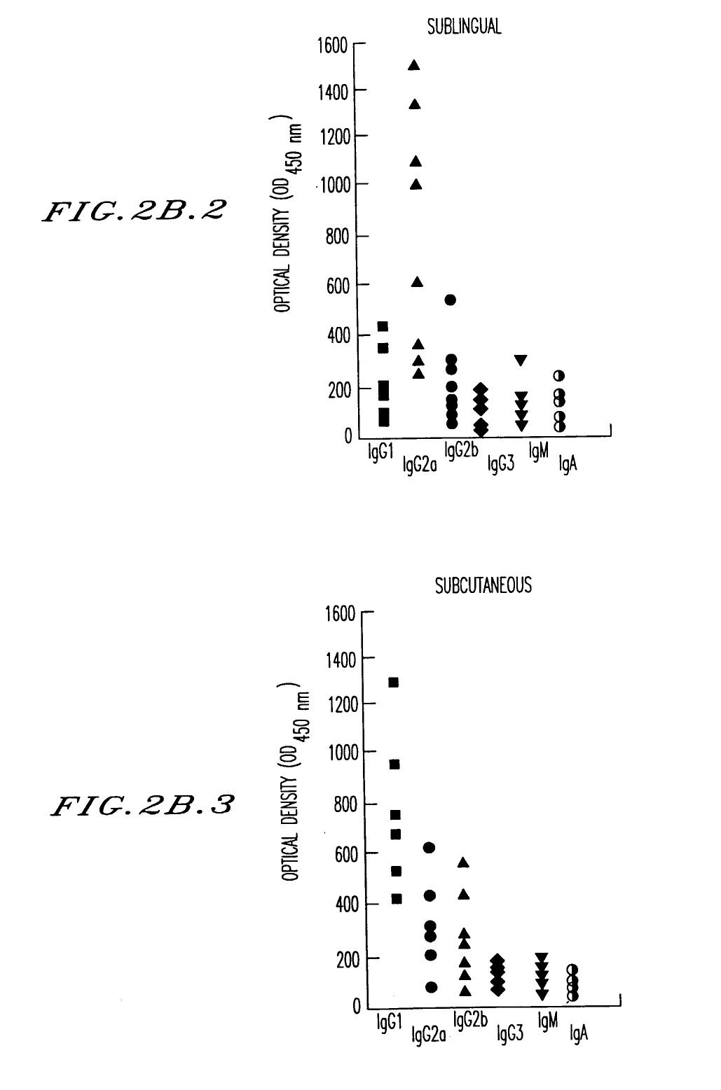

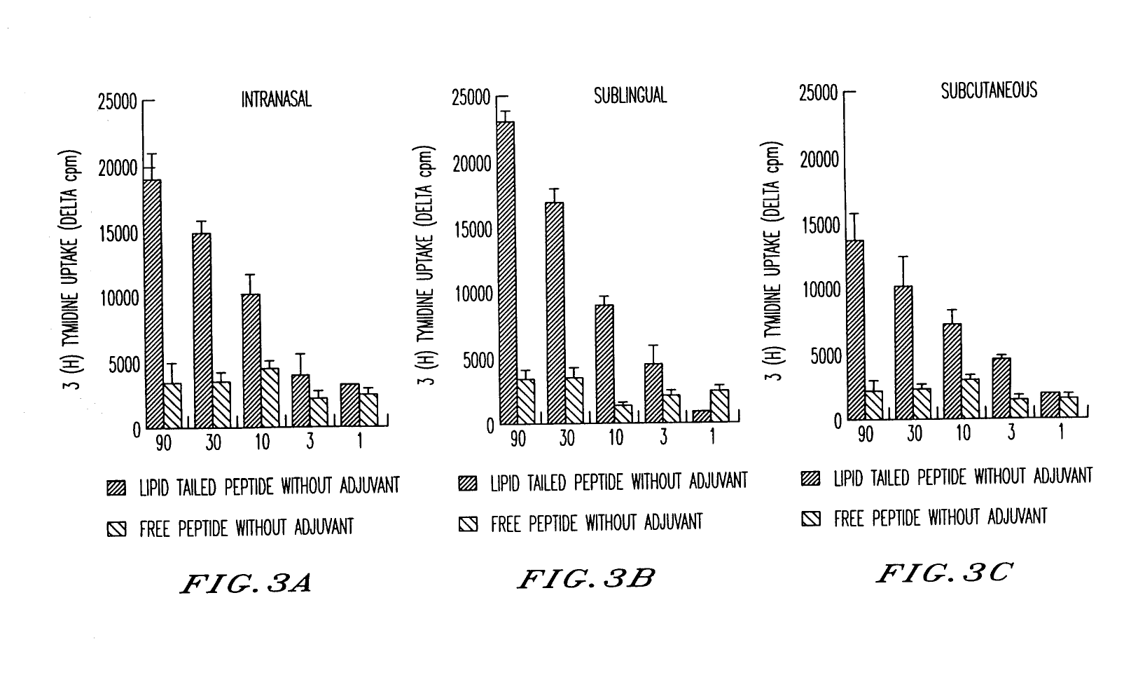

The Abs induced were also specific of parasite native proteins: the Abs produced by mucosally immunized BALB/c and C3H/HeJ were found to react with the intact parasite by IFAT, both at sporozoite and liver stages, thus demonstrating the biological relevance of this means of immunization(FIG. 2). They did not react with infected RBC's at different steps of intra-erythrocytic maturation, in agreement with the fact that LSA3 Ag is expressed in P. falciparum sporozoite and hepatic schizonts (Daubersies et coll., Nature Medicine 2000, col. 6, 11, 1258-1263).

These results clearly demonstrate that administration of a lipid-tailed polypeptide by the mucosal route effectively delivers the antigen to the immune system and shows that the lipid moiety is absolutely required.

Mucosal and Subcutaneous Routes Differ by the Profile of Isotypes Induced

In a second set of immunized animals, the determination of the isotype pattern revealed that both subcutaneous and mucosal routes induced predominantly antibodies of IgG1, IgG2a and IgG2b isotypes (FIG. 1B). However mucosal administration resulted in a preferential IgG2a serum antibody response, whereas the subcutaneous route was associated with a dominant IgG1 isotype response, and only a modest increase in total serum IgG2a (FIG. 1B) Anti-peptide IgA, IgM and IgG3 were detected at low and similar titers in all groups. The qualitative differences observed in the isotype distribution depending on the route used for immunization with the LSA3-NRII lipid-tailed polypeptide indicate that the transmucosal route would favor a Th1-like immune response.

Mucosal Administration of Lipid-tailed Polypeptides is Effective in Stimulating Systemic T Cell Responses in Lymph Node and Spleen Cells

As seen in FIG. 3, S.L. or I.N. applications of LSA3-NRII lipid tailed polypeptide, without a mucosal adjuvant, induced strong proliferative responses were found in spleen cells. Proliferative response was also detected in the inguinal lymph nodes of S.L. and I.N. immunized mice (maximal delta cpm=12525 in S.L. and 16189 in S.L) i.e. in T-cells taken a remote distance from the Ag delivery site. Surprisingly, using the same dose of LSA3-NRII lipid-tailed polypeptide we found that the proliferative responses of mucosally immunized animals were at higher levels compared to subcutaneously immunized animals (P<0.05). Proliferative responses were Ag-specific, as indicated by the lack of responses against an irrelevant polypeptide (polypeptide LSA1-J) (FIG. 3). In unimmunized control mice, or in control groups receiving the nonlipidated-polypeptide in the same dose range as the LSA3-NRII lipid-tailed polypeptide, weak or no proliferative responses were found, indicating that the lipid moiety was absolutely required (FIG. 3). Lymphoproliferative responses observed after mucosal administration were found to be of the CD4 phenotype as the responses were abrogated by antibodies against CD4 but not by anti-CD8 antibodies.

Mucosal Immunization Extends To Other Antigens:

To test whether the approach of vaccination via mucosal routes using lipid-tailed polypeptides might be generally applicable, LSA1-J lipid-tailed polypeptide, selected from LSA-1 Ag, (Fidock et coll., 1994) was delivered to BALB/c mice via transmucosal route. Intranasal and sub-lingual administration of LSA1-J lipid-tailed polypeptide was found to induce serum IgG responses in BALB/c mice (ELISA-RATIO range from 3.9 to 6.9), whereas the homologous non-lipidated polypeptide, without adjuvant, failed to induce any responses in these mice (ELISA-RATIO range from 0.3 to 0.7).

Similarly, the intranasal or sub-lingual administration of LSA1-J lipid-tailed polypeptide was found to induce strong T cell proliferative responses in spleen and lymph node cells from BALB/c mice (delta cpm range from =7698 to 10503), whereas the homologous non-lipidated polypeptide, without adjuvant, was inefficient (delta cpm range from 1632 to 2698). This result confirms that mucosal immunization by lipid-tailed polypeptides could be effective using other antigens.

T Lymphocyte Responses In Vitro After Immunization Via Mucosal Administration by Polypeptides TT-pol. (HIV):

BALB/c mice were immunized by mucosal administration (sublingual). The immunization dose was 100 μg. The antigen was polypeptide TT (tetanic toxin)-pol. (HIV) palmitic peptide. A palmitoyl residue is linked to a polypeptide consisting in part of the amino acid sequence of tetanus toxin and a peptide of pol gene of HIV-1, preferably a B cell epitope consisting in the sequence 476 to 484 of the Pol protein of HIV-1. The palmitoyl residue is covalently bound on the first Lysine (or K) of the tetanus toxin peptide sequence.

The present invention covers also a method for inducing an immune response in vivo comprising the administration of a composition containing a lipid-tailed polypeptide or peptide, said lipid-tailed polypeptide having at least a lipid residue bound to an epitope T amino acid sequence and optionally at least one epitope B amino acid sequence.

At day 15 ganglia sub-mandibulaires were collected. Making was accomplished by the CFSE technique (5,6-carboxyfluorescein diacetate succinimidyl ester or CFDA-SE) flourescent dye. This is a novel technique of monitoring in vivo CTL by labeling target cells with CFSE. This is a fluorescent cell proliferation marker used in combination with flow cytometry.

The CFSE technique can be used to determine kinetics of immune responses, track proliferation in minor subsets of cells and follow the acquisition of differentiation markers or internal proteins linked to cell division. Since its introduction in 1994 (Lyons, et al., J. Immunol. Methods 171 (1994) 131-137), the flow cytometric analysis of lymphocyte proliferation by serial halving of the fluorescence intensity of the vital dye CFSE (carboxyfluorescein diacetate, succinimidyl ester or CFDA-SE) has become widely used in immunological laboratories around the world.

The antigenic peptide used in this experiment had the following sequence:

H-K(PAM)TT-pol 476-484
Nh2-K(NεPam)GRQYIKANSKFIGITERGRILKEPVHGV-COOH

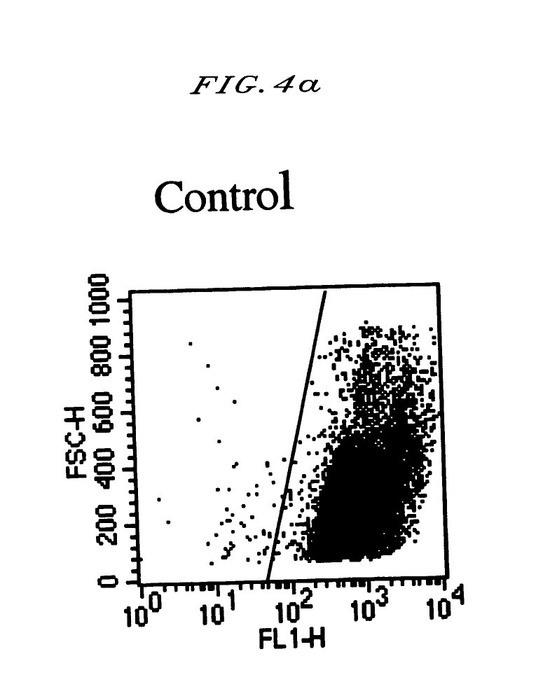

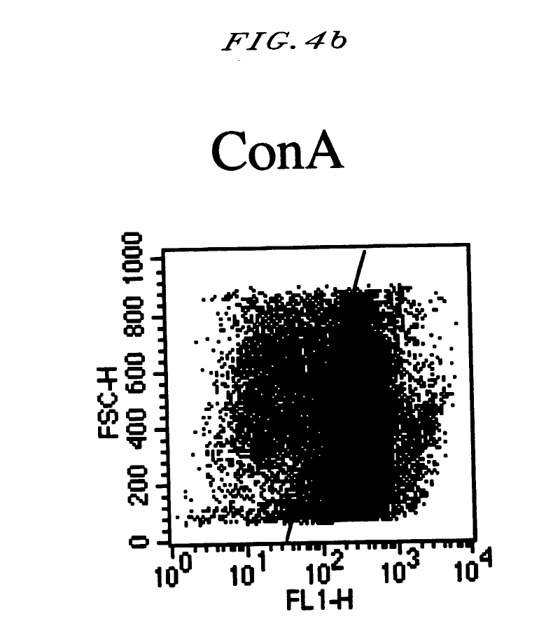

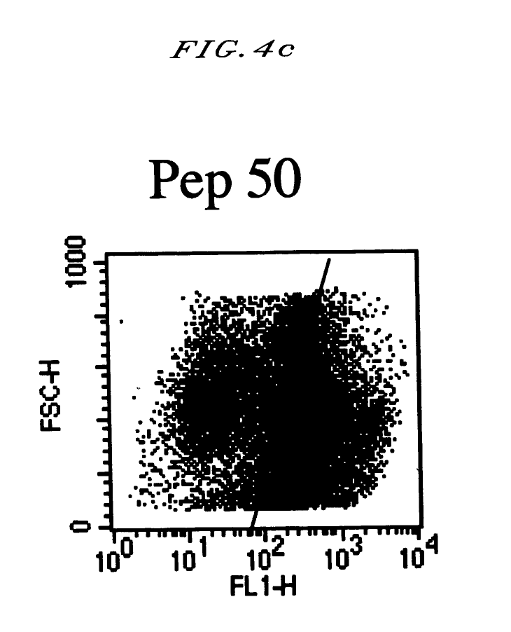

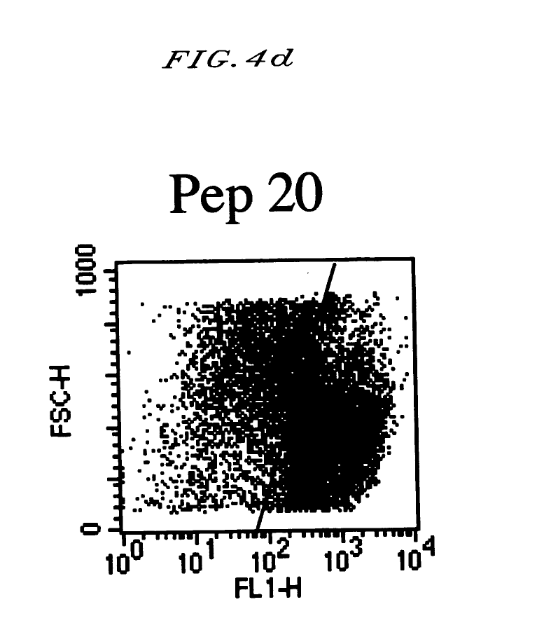

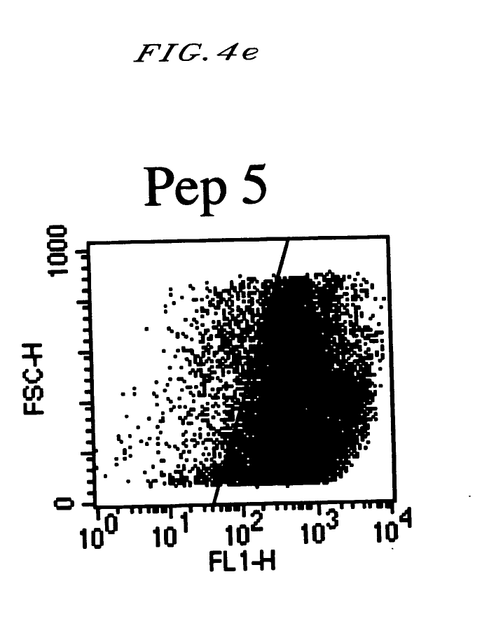

The results were obtain in vitro with 50, 20 and 5 μg of polypeptide. The results are presented in FIG. 4. The bar represents the threshold of significance. The shift towards the left of the bar represents the quantity of immune response obtained. The maximum immune response is given by the positive control CON A (Concanavaline A). As can be seen, the shift of fluorescence at 50 mg of peptide concentration is as intense as that provided by positive CON A control.

Discussion

Defining alternate routes of immunization is a current priority in vaccine research. Recently, the WHO Global Program for Vaccines and Immunization (GPV) highlighted that unsafe injections using unsterile needles, syringes or jet injectors, may transmit blood-borne infectious agents such as HIV and hepatitis viruses (Aylward et coll., 1995 ; Steinglass et coll, 1995). Mucosal delivery of the major pediatric vaccines has become an explicit goal of the Children's Vaccine Initiative of NIH as well as of WHO (Shepard et coll., 1995). Moreover vaccination is unfortunately not reliant purely on biotechnology but also on resources. Most of the vaccination programs are still very expensive and countries with the greatest demand are the least able to pay for them. It is well-known that the cost of equipment for delivering vaccines by parenteral routes (sterile syringes, needles, jet injectors. etc.) is, for GPVI vaccines, several times more expensive than the vaccines themselves (Shepard et coll., 1995; Hausdorff, 1996). The immunization, which usually require multiple injections necessitates well-trained and therefore expensive personal, and health-care infrastructures. In many populations and cultures, immunization using an application to mucosal surfaces would be more readily accepted than the physical penetration of needles (Holmgren, 1991). Hence a move from needle injection to mucosal application, which requires little, if any, special skill or equipment, would be positive from the economical, logistical, cultural and the safety points of view (Shepard et coll., 1995; King et coll., 1998).

Because they constitute a first-line defense system against pathogens, mucosal surfaces are particularly well-equipped in cells able to react to foreign antigens and process them. For example, it has been suggested that dendritic cells could play in the buccal epithelium a major role by engulfing Ags delivered with CTB and, after migrating to nearby lymph nodes, by presenting processed Ag to lymphocytes, prompting a strong immune response (Eriksson et coll., 1996). In theory, vaccines could be delivered to mucosal surfaces by the rectal, vaginal, conjuctival, oral, or nasal routes. However, not all the options are equally realistic. The rectal mucosa is well irrigated, but could be rejected by some cultures, and the vaginal mucosa not enough. Although Ags can be instilled into the conjuctival sac, they might elicit conjuctival inflammation, and occasionally infection. Thus, oral and nasal administrations may be the most practical options and are likely to be more readily accepted, particularly among children. The interest of intranasal immunization has been explored particularly for the induction of local, IgA-mediated, immunity. The sublingual route has been far less explored for immunization, although it offers over the intranasal route the interesting advantage of being not affected by local conditions such as rhinites due to colds or hayfever.

The lipidation of polypeptide and in a preferred embodiment the palmitoylation of polypeptide could induce a dramatic modification of the distribution of the lipid-tailed polypeptide within hydrophilic versus lipophylic compartments, resulting in a strong membrane interaction and relatively fast intracellular delivery(Thiiam, 99) which, indirectly, limits the extracellular proteolysis. The lipid moiety may also lead to increased release of pro-inflammatory cytokines by mucosal epithelial cells (Rouaix et coll., 1994).

In the present study, the determination of the Th and B-cell responses were used to probe the transmucosal delivery of Ags to the systemic immune system, with reference to the same parameters studied after parenteral immunization . Our findings suggest that the lipid-tailed antigens were actually delivered to immunocompetent cells after both nasal and sublingual administration, leading to the development of serum antibody production and to antigen-specific lymphoproliferative responses in the spleen and draining lymph nodes.

Strong B- and T-helper responses were induced after both parenteral or trans-mucosal routes depending upon the presence of the lipid-tail, the lymphoproliferative responses being even of higher intensities after intra-nasal or sublingual administration. Of particular interest were the qualitative differences observed in the antibody responses: using the same dose of lipopolypeptide, the mucosal immunization promoted preferentially an IgG2a response, while subcutaneous injection induced a dominant IgG1 isotype, suggesting that distinct antigen presenting cell populations were involved, depending on the immunization route. Hence, the method is not solely an exciting alternative to parenteral delivery of immunogens, but could also be used to preferentially channel immune responses towards the most effective type of response, depending on the target-pathogen.

The results presented herein validate the feasibility of systemic immunization by an antigen delivered through mucosal surfaces, by simple means and without adjuvant, at least for medium-size lipid-tailed polypeptides (as could be expected the same process proved also able, using HCMV derived polypeptides to efficiently induce CTL type of responses Ben Mohammed, manuscript in preparation). The extension of the mucosal immunization to larger, recombinant sub-unit vaccines could benefit from the development of chemical methods allowing regiospecific monoacylation of recombinants protein (Chopineau, 1998): The prospect of a vaccination protocol using spray, drops, aerosol, gels or sweets formulations is particularly attractive.

### EXAMPLES

Synthetic Lipid- and Non-lipid-tailed Polypeptides

The amino-Acid sequences were LSA3-NRII Ac-LEESQVNDDIFNSLVKSVQQEQQHNVK(Pam)NH2 and LSA1-J Ac-ERRAKEKLQEQQSDLEQRKADTKKK(Pam)NH2. in which the lipid-tail was covalently linked to the side chain of a C-terminal lysylamide residue. These lipid-tailed polypeptides were as previously described (Fidock et coll., 1994; BenMohamed et coil. 1997; Perlaza et coll., 1998). Most polypeptides and lipopeptides were >90% pure, as determined by HPLC.

Animals and Immunization

Groups of three to six BALB/c, C3H/HeJ, C57BL/6 mice, age 6-8 weeks, (Janvier), were given lipid-tailed polypeptides on days 0 and 14, using the sub-lingual or intranasal routes. For intranasal administration, 30 ml of sterile phosphate-buffered saline (PBS), containing 100 mg of lipopeptide, were distributed equally in both nares (15 ml in each nostril) using a 100 ml sterile pipette tip. The pipette tips were not placed into the nares in order to avoid localized trauma. For sub-lingual administration, cotton wool was soaked in 30 ml of sterile phosphate-buffered saline (PBS), containing 100 mg of lipopeptide, and then applied to the buccal cavity (sub-lingual area) for 20 to 30 min. Control mice were injected sub-cutaneously with 100 ml of sterile phosphate-buffered saline (PBS), containing 100 mg lipid-tailed polypeptide using a sterile 1 ml syringe. To investigate if the palmitic acid moiety plays a role in the systemic immunogenicity of lipid-tailed polypeptides, free analogous polypeptides were used as controls.

Detection of Serum Antibody Responses

Individual blood samples were obtained via the retro-orbital plexus by 9 to 15 days post immunization (dpi) and sera were stored at-70ᵢ C until assayed for IgG, IgA and IgM polypeptide- and parasite-specific Abs. The presence of anti-peptide antibodies in sera was determined using Enzyme-linked immunosorbent assay (ELISA) as reported previously (BenMohamed et coll., 1997). ELISA plates (Nunc, Roskilde, Denmark) were coated overnight at 4ᵢ C with 0.1 ml of LSA3-NRII or LSA1-J polypeptide solution at 3 zg/ml in PBS buffer pH 7.4 containing 3% BSA. The LSA1-J polypeptide was used as the irrelevant control of LSA3-NRII and vice versa. The plates were washed twice in PBS with 0.01% Tween-20 (PBS-T), blocked for 1 hr in PBS-T supplemented with 1% BSA prior to the addition of 0.1 ml of 1/100 dilution of mouse sera. The plates were then incubated at 37ᵢC for one hour. After washing, the bound IgG were detected using peroxidase-conjugated goat anti-mouse IgG (Biosys, Compiegne, France) added at a 1/2000 dilution. Following incubation at 37ᵢC for 1 hour and a final wash, 50 μl of 0.30% H₂O₂ containing orthophenylenediamine dihydrochloride (OPD, Sigma, St. Louis), dissolved in 0.1 M citrate buffer (pH 5.0) were added to each well at room temperature. The OD450 nm was measured using a multichannel spectrophotometer (Titertek Multiskan MCC. 340). Individual sera from all groups were diluted 1/100 and analyzed separately. Preimmune sera were used as negative controls and the results were expressed either as optical density (OD) at 450nm or as ELISA-RATIO calculated as follows: OD450 nm postimmune sera divided by OD450 nm preimmune sera. For polypeptide-specific ELISAs, sample dilution were considered positive if the OD450 nm recorded for that dilution was at least twofold higher than the OD450 nm recorded for a naive sample at the same dilution (Fidock et coll., 1994 ; Bottius et coll., 1996). Isotype analysis of mouse was carried out using class specific alkaline phosphatase-conjugated Goat anti-Mouse IgA, IgM, IgGI, IgG2a, IgG2b or IgG3 HRP-Labeled (Southern Biotechnology Associates, Birmingham, USA) added at a 1/2000 dilution in PBS-T, as previously described (BenMohamed et coll., 1997).

Immunofluorescent Ab Assay

The reactivity of the sera against native proteins from various stages of the parasite were analyzed by IFAT using either (i) P. falciparum NF54 strain sporozoites (a gift of W. Eling), or (ii) sections from liver biopsies containing day 5 P. falciparum liver schizonts [42], or (iii) day 6 ½ P. falciparum liver schizonts obtained from a Chimpanzee [43]. IFAT-labeled anti-human IgG, -A, -M (Diagnostic Pasteur France) or anti-mouse (Cappel. Wester Chester. PA) diluted 1/200 were employed as second Abs.

Lymphocyte Proliferative Assay

For proliferation assays, spleen and inguinal lymph nodes were obtained from mice (3 to 6 per group) on 14 dpi using sterile forceps and placed into ice-cold Hank's balanced salt solution (HBSS). Single-cell suspensions were prepared by crushing the tissues between the frosted ends of two microscope slides. Red blood cells were removed by treatment with ammonium chloride on ice for 10 min. The single-cell suspensions were washed twice in RPMI-1640 (Gibco, Courbevoie, France) and were adjusted to 4×106 cells/ml in RPMI-1640 media supplemented with 1.5% heat-inactivated fetal calf serum (FCS), 1% penicillin-streptomycin, 1% glutamine, 5.10⁻⁵ M 2μ-mercaptoethanol (Gibco), and 1% N-n-hydroxyethylpiperizine-N′-2 ethanesulphon acid (HEPES), pH 7.4, and used as previously described (BenMohamed et coll., 1997). Equal volumes of cells and complete medium or complete medium with LSA3-NRII or LSA1-J polypeptides were mixed to give a final concentration of 2×106 cells/ml in medium alone or in medium with polypeptide at 90, 30, 10, 3, or 1 mg/ml. The cell suspensions were incubated for 72h at 37° C. and 7.5% CO₂. Three days lather, one μCi of tritiated deoxythymidine ((3H)TdR) (Amersham, Les Ulis, France) was added to each well, for 16h before the cultures were harvested (Skatron, Lierbyen, Norway) and the incorporated radioactivity determined by liquid scintillation (LKB-Wallac, Turku, Finland). Results are expressed as the mean cpm of cell-associated (3H)TdR recovered from wells containing Ag, substracted by the mean cpm of cell-associated (3H)TdR recovered from wells without Ag (D cpm) (average of triplicates). The results were considered positive when the D cpm is ≧ to 1000 cpm and stimulation index >2 (Fidock et coll., 1994 ; Bottius et coll., 1996 ; BenMohamed et coll., 1997).

Statistical Analysis

Figures and tables represent data from one of at least two independent experiments.

The data are expressed as the mean±SEM and compared by using Student's t test. The results were analyzed by using the STATVIEW II statistical program (Abacus Concepts, Berkeley, CA) on a Macintosh computer and were considered statistically significant if P values were less than 0.05.

### REFERENCES

1. Alving C R, Koulchin V, Glenn G M, Rao M. 1995. Liposomes as carriers of peptide antigens: induction of antibodies and cytotoxic T lymphocytes to conjugated and unconjugated peptides. Immunol Rev 1995 Jun; 145:5-31

2. Aylward B, Kane M, McNair-Scott R, Hu D J. Model-based estimates of the risk of human immunodeficiency virus and hepatitis B virus transmission through unsafe injections. 1995. Int J Epidemiol. 24(2):446-52. Published erratum appears in Int J Epidemiol 1996 Jun;25(3):688

3. Ball, J M., Hardy, M E., Atmar, R L., Conner, M E. and Estes M K. 1998. Oral immunization with recombinant Norwalk virus-like particles induces a systemic and mucosal immune response in mice. J Virol. 72(2):1345-53

4. BenMohamed L, Gras-Masse H, Tartar A, Daubersies P, Brahimi K, Bossus M, Thomas A, Druilhe P. 1997. Lipopeptide immunization without adjuvant induces potent and long-lasting B, T helper, and cytotoxic T lymphocyte responses against a malaria liver stage antigen in mice and chimpanzees. Eur J Immunol 1997. 27(5):1242-53

5. Bottius E, BenMohamed L, Brahimi K, Gras H, Lepers J P, Raharimalala L, Aikawa M, Meis J, Slierendregt B, Tartar A, Thomas A, Druilhe P. 1996. A novel Plasmodium falciparum sporozoite and liver stage antigen (SALSA) defines major B, T helper, and CTL epitopes. J Immunol 1996 Apr 15;156(8):2874-84

6. Bourgault I, Chirat F, Tartar A, Levy J P, Guillet J G, Venet A. 1994. Simian immunodeficiency virus as a model for vaccination against HIV. Induction in rhesus macaques of GAG- or NEF-specific cytotoxic T lymphocytes by lipopeptides. J Immunol. 152(5):2530-7

7. Bourgault-Villada, I. Mortara, L. Aubertin, A. M. Gras-Masse, H. Levy, J. P. and J. G. Guillet. 1997. Positive role of macaque cytotoxic T lymphocytes during SIV infection : decrease of cellular viremia and increase of asymptomatic clinical period FEMS Imnmunology and Medical Microbiology, 19, 81-87.

8. Chopineau J; Robert S; Fenart L; Cecchelli R; Lagoutte B; Paitier S; Dehouck M P and D. Domurado. 1998. Monoacylation of ribonuclease A enables its transport across an in vitro model of the blood-brain barrier. J Controlled Release, 56:1-3, 231-7

9. Deres K, Schild H, Wiesmuller K H, Jung G, Rammensee H G. 1989. In vivo priming of virus-specific cytotoxic T lymphocytes with synthetic lipopeptide vaccine. Nature. 342(6249):561-4

10. Di Tommaso A, Saletti G, Pizza M, Rappuoli R, Dougan G, Abrignani S, Douce G, De Magistris MT. 1996. Induction of antigen-specific antibodies in vaginal secretions by using a nontoxic mutant of heat-labile enterotoxin as a mucosal adjuvant. Infect Immun. 64(3):974-9

11. Eriksson K, Ahlfors E, George-Chandy A, Kaiserlian D, Czerkinsky C. 1996. Antigen presentation in the murine oral epithelium. Immunology. 88(1):147-52

12. Fidock D A, Gras-Masse H, Lepers J P, Brahimi K, Benmohamed L, Mellouk S, Guerin-Marchand C, Londono A, Raharimalala L, Meis J F, and Druilhe, P. 1994. Plasmodium falciparum liver stage antigen-I is well conserved and contains potent B and T cell determinants. J Immunol. 153(1):190-204

13. Foldvari, M., Attah-Poku, S., Hu, J., Li, Q., Hugues, H., Babiuk, L. A. and S. Kruger. 1998. Palmitoyl derivatives of interferon alpha: potential for cutaneous delivery J. Pharm Sci. 87 (10) 1203-8.

14. Fynan E F, Webster R G, Fuller D H, Haynes J R, Santoro J C, Robinson H L. 1993. DNA vaccines: protective immunizations by parenteral, mucosal, and gene-gun inoculations. Proc Natl Acad Sci U S A. 90(24): 11478-82

15. Gonzalez C, Hone D, Noriega F R, Tacket C O, Davis J R, Losonsky G, Nataro J P, Hoffinan S, Malik A, Nardin E. 1994. Salmonella typhi vaccine strain CVD 908 expressing the circumsporozoitc protein of Plasmodium falciparum: strain construction and safety and immunogenicity in humans. J Infect Dis. 169(4):927-31

16. Gould-Fogerite S, Edghill-Smith Y, Kheiri M, Wang Z, Das K, Feketeova E, Canki M, Mannino R J. 1994. Lipid matrix-based subunit vaccines: a structure-function approach to oral and parenteral immunization. AIDS Res Hum Retroviruses 1994; 10 Suppl 2:S99-103

17. Gregoriadis G. 1995. Engineering liposomes for drug delivery: progress and problems. Trends Biotechnol 1995 Dec; 13(12) :527-37

18. Hantman M J., Hohmann, E., Murphy, C G., Knipe, D M., Miller, S I. (1999). Antigen delivery systems: Development of recombinant live vaccines using viral or bacterial vectors. ‘In Handbook of Mucosal Immunology’, (P L. Ogra, J. Mestecky, M E. Lamm, W. Strober, J. Bienenstock, and McGhee, J R. eds.) pp779-791. Academic Press, San Diego.

19. Hausdorff W P. 1996. Prospects for the use of new vaccines in developing countries: cost is not the only impediment. Vaccine. 14(13):1179-86

20. Holmgren J. 1991. Mucosal immunity and vaccination. FEMS Microbiol Immunol. 4(1):1-9

21. Holmgren J, Brantzaeg P, Capron A, Francotte M, Kilian M, Kraehenbuhl J P, Lehner T, Seljelid R. 1996. European Commission COST/STD Initiative. Report of the expert panel VI. Concerted efforts in the field of mucosal immunology. Vaccine 1996 May;14(7) :644-64

22. King J C Jr, Lagos R, Bernstein D I, Piedra P A, Kotloff K, Bryant M, Cho I, Belshe R B. 1998. Safety and immunogenicity of low and high doses of trivalent live cold-adapted influenza vaccine administered intranasally as drops or spray to healthy children. J Infect Dis. 177(5):1394-7

23. Klavinskis L S, Bamfield C, Gao L, Parker S. 1999. Intranasal immunization with plasmid DNA-lipid complexes elicits mucosal immunity in the female genital and rectal tracts. J Immunol 1999 Jan 1;162(1):254-62

24. Levi R, Aboud-Pirak E, Leclerc C, Lowell G H, Arnon R. 1995. Intranasal immunization of mice against influenza with synthetic peptides anchored to proteosomes. Vaccine 1995 Oct;13(14):1353-9

25. Levine M M, Dougan G. 1998. Optimism over vaccines administered via mucosal surfaces. Lancet. 351(9113): 1375-6

26. Livingston B D, Crimi C, Grey H, Ishioka G, Chisari F V, Fikes J, Grey H, Chesnut R W, Sette A. 1997. The hepatitis B virus-specific CTL responses induced in humans by lipopeptide vaccination are comparable to those elicited by acute viral infection. J Immunol. 159(3): 1383-92

27. Mannino R J, Gould-Fogerite S. 1995. Lipid matrix-based vaccines for mucosal and systemic immunization. Pharm Biotechnol. 6:363-87

28. Martinon F, Gras-Masse H, Boutillon C, Chirat F, Deprez B, Guillet J G, Gomard E, Tartar A, Levy J P. 1992. Immunization of mice with lipopeptides bypasses the prerequisite for adjuvant. Immune response of BALB/c mice to human immunodeficiency virus envelope glycoprotein. J Immunol. 149(10):3416-22

29. Mestecky J, Michalek S M, Moldoveanu Z, Russell M W. 1997. Routes of immunization and antigen delivery systems for optimal mucosal immune responses in humans. Behring Inst Mitt. (98):33-43

30. Metzger J, Jung G, Bessler W G, Hoffinann P, Strecker M, Lieberknecht A, Schmidt U. 1991. Lipopeptides containing 2-(palmitoylamino)-6,7-bis(palmitoyloxy) heptanoic acid: synthesis, stereospecific stimulation of B-lymphocytes and macrophages, and adjuvanticity in vivo and in vitro. J Med Chem. 34(7):1969-74

31. Michalek S M, O'Hagan D T, Gould-Fogerite, S. Rimmelzwaan, G F. Osterhaus, A D. (1999). Antigen delivery systems: Nonliving Microparticles, Liposomes, Cochleates and ISCOMs. “In Handbook of Mucosal Immunology”, (P L. Ogra, J. Mestecky, M E. Lamm, W. Strober, J. Bienenstock, and McGhee, J R. eds.) pp759-779. Academic Press, San Diego.

32. Mora A L, Tam J P. 1998. Controlled lipidation and encapsulation of peptides as a useful approach to mucosal immunizations. J Immunol. 161(7):3616-23

33. Mortara, L. Letourneur, F. Gras-Masse, H. Venet, A. Guillet, J. G. I. Bourgault-Villada. 1998. Selection of Virus Variants and Emergence of Virus Escape Mutants after Immunization with an Epitope Vaccine. Journal of Virology. 1403-1410

34. Nardelli B, Haser P B, Tam J P. 1994. Oral administration of an antigenic synthetic lipopeptide (MAP-P3C) evokes salivary antibodies and systemic humoral and cellular responses. Vaccine. 12(14):1335-9

35. Nataro J P., Levine, M M. (1999). Enteric bacterial vaccine: Salmonella, Shigella, Cholera, Escherichia coli. “In Handbook of Mucosal Immunology”, (P L. Ogra, J. Mestecky, M E. Lamm, W. Strober, J. Bienenstock, and McGhee, J R. eds.) pp851-865. Academic Press, San Diego.

36. O'Hagan D T, Jeffery H, Davis S S. 1994. Long-term antibody responses in mice following subcutaneous immunization with ovalbumin entrapped in biodegradable microparticles. Vaccine 1993;1 1(9):965-9

37. Perlaza B L, Arevalo-Herrera M, Brahimi K, Quintero G, Palomino J C, Gras-Masse H, Tartar A, Druilhe P, Herrera S. 1998. Immunogenicity of four Plasmodium falciparum preerythrocytic antigens in Aotus lemurinus monkeys. Infect Immun. 66(7): 3423-8

38. Porgador A, Staats H F, Faiola B, Gilboa E, Palker T J. 1997. Intranasal immunization with CTL epitope peptides from HIV-1 or ovalbumin and the mucosal adjuvant cholera toxin induces peptide-specific CTLs and protection against tumor development in vivo. J Immunol. 158(2):834-41

39. Ramani K, Hassan Q, Venkaiah B, Hasnain S E, Sarkar D P. 1998. Site-specific gene delivery in vivo through engineered Sendai viral envelopes. Proc Natl Acad Sci U S A. 29;95(20): 11886-90

40. Rouaix F, Gras-Masse H, Mazingue C, Diesis E, Ridel P R, Estaquier J, Capron A, Tartar A, Auriault C. 1994. Effect of a lipopeptidic formulation on macrophage activation and peptide presentation to T cells. Vaccine. 12(13):1209-14

41. Sabin A B. 1985. Oral poliovirus vaccine: history of its development and use and current challenge to eliminate poliomyelitis from the world. J Infect Dis, 151(3):420-36

42. Shepard D S, Walsh J A, Kleinau E, Stansfield S, Bhalotra S. 1995. Setting priorities for the Children's Vaccine Initiative: a cost-effectiveness approach. Vaccine 1995;13(8):707-14

43. Snider D P. The mucosal adjuvant activities of ADP-ribosylating bacterial enterotoxins. 1995. Crit Rev Immunol. 15(3-4):317-48

44. Staats H F, Jackson R J, Marinaro M, Takahashi I, Kiyono H, McGhee J R. 1994. Mucosal immunity to infection with implications for vaccine development. Curr Opin Immunol. 6(4):572-83

45. Steinglass R, Boyd D, Grabowsky M, Laghari A G, Khan M A, Qavi A, Evans P. 1995. Safety, effectiveness and ease of use of a non-reusable syringe in a developing country immunization programme. Bull World Health Organ. 73(1):57-63

46. Stephens G; O'Luanaigh N; Reilly D; Harriott P; Walker B; Fitzgerald D. and N. A. Moran. 1998. sequence within the cytoplasmic tail of Gpllb independently activates platelet aggregation and thromboxane synthesis. J Biol Chem, 273:32, 20317-22.

47. Takahashi H, Takeshita T, Morein B, Putney S, Germain RN, Berzofsky J A. 1990. Induction of CD8+ cytotoxic T cells by immunization with purified HIV-1 envelope protein in ISCOMs. Nature. 344(6269):873-5

48. Thiam, K. Loing, E. Gilles, F. Verwaerde, C. Quatannens, B. Auriault, C. and H. Gras-Masse. Induction of apoptosis by Protein Kinase C pseudosubstrate lipopeptides in Several Human cells. Letters in Peptide Sciences. (1997) 4-397-402

49. Thiam K, Loing E, Delanoye A, Diesis E, Gras-Masse H, Auriault C, Verwaerde C. Unrestricted agonist activity on murine and human cells of a lipopeptide derived from IFN-gamma. 1998. Biochem Biophys Res Commun 1998 Dec 30;253(3):639-47 THIAM Kader, LOING Estelle, VER WAERDE Claudie, AURIAULT Claude, and GRAS-MASSEHélene‡*.IFN-g derived lipopeptides: Influence of the lipid modification on the conformation and on the ability to induce MHC class II expression in Murine and Human cells (J. Med. Chem. in press)1999

50. Tsunoda, L. Sette, A. Fujinami, R. Oseroff, C. Ruppert, J. Dahlberg, C. Southwood, S. Arrhenius, T. Kuang, L-Q. Kubo, R. T. Chesnut, R. W. and G. Y. Ishioka. 1999. Lipopeptide particles as the immunologically active component of CTL inducing vaccines. Vaccine Vol. 17 (7-8) pp. 675-685

51. Smith R E, Donachie A M, Mowat A M. 1998. Immune stimulating complexes as mucosal vaccines. Immunol Cell Biol 1998 Jun;76(3):263-9

52. Uhl B, Wolf B, Schwinde A, Metzger J, Jung G, Bessler W G, Hauschildt S. 1991. Intracellular localization of a lipopeptide macrophage activator: immunocytochemical investigations and EELS analysis on ultrathin cryosections of bone marrow-derived macrophages. J Leukoc Biol. 50(1): 10-8

53. Uhl B, Speth V, Wolf B, Jung G, Bessler W G, Hauschildt S. 1991. Rapid alterations in the plasma membrane structure of macrophages stimulated with bacterial lipopeptides. Eur J Cell Biol. 58(1):90-8

54. Vitiello A, Ishioka G, Grey H M, Rose R, Famess P, LaFond R, Yuan L, Chisari F V, Furze J, Bartholomeuz R. 1995. Development of a lipopeptide-based therapeutic vaccine to treat chronic. HBV infection. I. Induction of a primary cytotoxic T lymphocyte response in humans. J Clin Invest. 95(1):341-9

55. Yamamoto Shingo, Hiroshi Kiyono, Masafumi Yamamoto, Koichi Imaoka, Miho Yamamoto, Kohtaro Fujihashi, Frederik W. Van Ginkel, Masatoshi Noda, Yoshifumi Takeda, and Jerry R. McGhee. 1997. A nontoxic mutant of cholera toxin elicits Th2-type responses for enhanced mucosal immunity (cholera toxin mutant/Th1 and Th2 subsets/mucosal immunity). Proc. Natl. Acad. Sci. USA. Vol. 94, pp. 5267-5272.

Obviously, numerous modifications and variations of the present invention are possible in light of the above teachings. It is therefore to be understood that within the scope of the appended claims, the invention may be practiced otherwise than as specifically described herein.

All publications cited herein are incorporated herein by reference in their entirety unless otherwise noted.

1. A method of inducing an immune response by the delivering of an effective amount of lipid-tailed protein to a mucosal membrane of a subject.

2. The method of claim 1, wherein the lipoprotein is applied to the mucosal membrane without adjuvant.

3. The method of claim 1, wherein the lipoprotein is applied to the mucosal membrane without using a needle.

4. The method of claim 1, wherein the lipoprotein is applied intranasally, sub-lingually, by eye-drops, or suppositories.

5. The method of claim 1, wherein the lipoprotein has at least one lipid coupled to a functional group of the said protein.

6. The method of claim 1, wherein the lipoprotein has at least one lipid coupled to a α-NH₂ and/or ε-NH₂ functional group of the peptide.

7. The method of claim 1, wherein application of the lipoprotein induces a B cell response.

8. The method of claim 1, wherein application of the lipoprotein induces a T cell response.

9. The method of claim 1, wherein application of the lipoprotein induces a systemic B and/or T cell response.

10. A composition consisting in at least one lipoprotein inducing a mucosal immune response in vivo in absence of toxic adjuvant.

11. A composition according to claim 10, wherein the adjuvant is non-toxic for the mucosal membranes.

12. A lipopeptide, wherein the lipopeptide is tailed with a lipid component.

13. The lipopeptide of claim 11, wherein the lipid component is a palmitoyl residue having 16 carbon atoms.

14. The lipopeptide of claim 12, wherein the lipopeptide is:

| | |
|---|---|
| LSA3-NRII | Ac-LEESQVNDDIFNSLVKSVQQEQQHNVK(PAM)NH2 OR |
| LSA1-J | Ac-ERRAKEKLQEQQSDLEQRKADTKKK(PAM). |

15. The method of claim 9, wherein the lipopetide is:

| | |
|---|---|
| LSA3-NRII | Ac-LEESQVNDDIFNSLVKSVQQEQQHNVK(PAM)NH2 OR |
| LSA1-J | Ac-ERRAKEKLQEQQSDLEQRKADTKKK(PAM)NH2. |

16. A composition consisting in at least one lipopeptide inducing a mucosal immune response in vivo in the absence of toxic adjuvant, wherein the lipopeptide is at least one lipopeptide according to claim 13.

17. A vaccine composition for mucosal administration containing at least one lipopeptide inducing an B and/or T cell response in vivo in absence of adjuvant.

18. A vaccine composition containing a lipopeptide according to claim 13 in the absence of adjuvant.

19. An immunogenic composition containing a lipopeptide according to claim 13.

20. A method of stimulating T-Lymphocyte responses in vitro after immunization via mucosal administration comprising the following steps:
a) immunizing BALB/C mice by mucosal administration with a peptide tetanic toxin-pol HIV palmitic antigen,
b) collecting of ganglia sub-mandibulaires at day 15, and
c) visualizing T cell responses by labeling target cells with CFSE.

21. The method of claim 1, further comprising administering a composition containing a lipid-tailed polypeptide or peptide, said lipid-tailed peptide having at least a lipid residue bound to an epitope T amino acid sequence and optionally at least one epitope B amino acid sequence.

22. The method of claim 21, wherein the lipopeptide is an antigenic lipopeptide of sequence:
H-K(PAM)TT-pol 476-484
Nh2-K(NεPam)GRQYIKKANSKFIGITERGRILKEP-COOH.

23. The method of claim 1, wherein the lipopeptide is a lipid-tailed epitope T.

24. The method of claim 23, wherein the lipopeptide is a lipid-tailed epitope T covalently linked to an epitope B peptide.

25. A composition comprising lipid-tailed polypeptide or peptide, said lipid-tailed peptide having at least a lipid residue bound to an epitope T amino acid sequence and optionally at least one epitope B amino acid sequence.
